(19) United States
(12) Patent Application Publication
(10) Pub. No.: US 2026/0266939 A1
(43) Pub. Date: Sep. 10, 2026

(54) CORRECTING THE QUANTIFICATION BIAS IN MAGNETIZATION-PREPARED IMAGING

(71) Applicant: THE JOHNS HOPKINS UNIVERSITY, Baltimore, MD (US)

(72) Inventors: Qin Qin, Baltimore, MD (US); Dan Zhu, Baltimore, MD (US)

(21) Appl. No.: 19/164,093

(22) PCT Filed: Mar. 11, 2024
(86) PCT No.: PCT/US2024/019405

§ 371 Date: Sep. 10, 2025

Related U.S. Application Data
(60) Provisional application No. 63/489,635, filed on Mar. 10, 2023.

(51) Int. Cl. G01R 33/56; G01R 33/561; G06T 5/50

(52) U.S. Cl. CPC G01R 33/5602; G01R 33/5614; G01R 33/5615; G06T 5/50; G06T 2207/10088

(57) ABSTRACT

To correct the quantification bias in magnetization-prepared imaging, an additional scan is added after the saturation module and immediately followed by the same readout, in order to record the bias from the k-space filtering effect induced by the T₁ relaxation during the long echo train. This bias is subtracted using complex data from the other images with magnetization preparation modules of interest.

### CROSS REFERENCE TO RELATED APPLICATIONS

This application claims the benefit of U.S. Provisional Patent Application No. 63/489,635 filed on, Mar. 10, 2023, which is incorporated by reference, herein, in its entirety.

### GOVERNMENT SUPPORT

The present invention was made with government support under grant numbers HD103538, EB031771, HL138182, and HL144751 awarded by the National Institutes of Health. The government has certain rights in the present invention.

### FIELD OF THE INVENTION

The present invention relates generally to medical imaging. More particularly, the present invention relates to systems and methods for correcting the quantification bias in magnetization-prepared imaging.

### BACKGROUND OF THE INVENTION

Due to the proximity between the shorter RF wavelength at higher field strength and the size of the tissue, the homogeneity of the transmit RF field (B₁⁺) is affected by both tissue composition and body geometry. Rapid measurement of the spatial variation of the flip angles (FA) for the organs of interest is desired for many MRI applications, including subject-adaptive RF shimming using multiple RF transmission channels, FA correction for steady-state based T₁ or T₂ measurement and spectroscopy-based metabolite quantification, determining the electric properties of the tissue or the local SAR distribution, or evaluating the sensitivity to B₁⁺ field inhomogeneities for novel pulse sequences. A number of B₁⁺ mapping approaches have been commercialized by vendors. The conventional double-angle method (DAM), by taking the ratio of two images with excitation FA=[α, 2α] for gradient echo or excitation/refocusing FA=[α/2α, 2α/4α] for spin echo acquisitions, requested TR>>T₁ in order to remove the T₁ effect, thus is very time consuming. The saturated double-angle method (SDAM) alleviated this TR restriction by inserting a B₁-insensitive saturation module with a fixed saturation delay. Both DAM and SDAM are based on multislice 2D imaging, which is affected by imperfect slice profiles due to RF pulse shapes or B₀ inhomogeneities. To avoid the dependence of the excitation profile across each slice, the so called actual flip-angle imaging (AFI) method employs 3D fast gradient echo acquisition in pulsed steady-state with two interleaved TRs and the same FA. In contrast to these magnitude-based techniques, a phase-based method utilizes the Bloch-Siegert shift to encode the B₁ information. Both AFI and Bloch-Siegert methods are still rather slow, taking minutes to cover a large volume.

It would therefore be advantageous to provide a system and method for correcting the quantification bias in magnetization-prepared rapid gradient echo imaging.

### SUMMARY OF THE INVENTION

The foregoing needs are met by the present invention which provides a method for magnetic resonance imaging including a scan. The scan includes an acquisition scheme. The acquisition scheme of the scan is applied immediately after a saturation module in order to obtain the imaging bias induced by a T₁ relaxation during the long readout.

In accordance with an aspect of the present invention the scan can take the form of a FLASH or bSSSP acquisition. The T₁ relaxation is during a long echo train. The imaging bias is subtracted using data from other images with pre-selected preparation modules.

In accordance with an aspect of the present invention, a method for magnetic resonance imaging of a subject including a first scan comprising a B₁-insensitive saturation module followed by a fixed saturation delay to generate a consistent magnetization state, a 3D FLASH acquisition with low-high profile ordering, and an RF-prep module with a short hard pulse (FA=α) followed by FLASH readout to generate a FA-encoded image. The method comprises a second scan comprising a B₁-insensitive saturation module followed by a fixed saturation delay to generate a consistent magnetization state, a 3D FLASH acquisition with low-high profile ordering, and acquiring a normalizing image without applying the RF-prep module. The method also comprises a third scan comprising a 3D FLASH acquisition with low-high profile ordering, wherein the FLASH acquisition is applied immediately after the saturation module in order to obtain the imaging bias induced by the T₁ relaxation during the long readout.

In accordance with an aspect of the present invention a system for magnetic resonance imaging of a subject includes a magnetic resonance imaging device. The system also includes a computer processing device configured for communication and control of the magnetic resonance imaging device. The computer processing device is programmed for executing the following steps: a first scan taking the form of a B₁-insensitive saturation module followed by a fixed saturation delay to generate a consistent magnetization state, a 3D FLASH acquisition with low-high profile ordering, and an RF-prep module with a short hard pulse (FA=α) followed by FLASH readout to generate a FA-encoded image; a second scan taking the form of a B₁-insensitive saturation module followed by a fixed saturation delay to generate a consistent magnetization state, a 3D FLASH acquisition with low-high profile ordering, and acquiring a normalizing image without applying the RF-prep module; and a third scan taking the form of a 3D FLASH acquisition with low-high profile ordering, wherein the FLASH acquisition is applied immediately after the saturation module in order to obtain the imaging bias induced by the T₁ relaxation during the long readout.

In accordance with another aspect of the present invention, the processing device is programmed for subtracting the imaging bias from the first and the second scans and conducting an RF-prepared method for FA-encoding and normalizing the first and second scans. The B₁-insensitive saturation module being used to establish Mₙₒᵣₘ after saturation recovery during a fixed delay. The third scan can be used to obtain intercept, b, without the saturation delay of a normalizing scan. The long readout comprises a long echo train. The imaging bias is subtracted using data from other images with pre-selected preparation modules. The imaging bias is from the k-space filtering effect induced by the T₁ relaxation.

### BRIEF DESCRIPTION OF THE DRAWINGS

FIG. 1 illustrates a schematic of a proposed image acquisition protocol of the present invention.

FIG. 2 illustrates image views evaluating the 3D RF-prepared B₁ mapping technique in the brain. The third saturated image added a very short extra time (1.54 s acquisition duration for the single-shot readout), and revealed this non-negligible bias.

FIG. 3 illustrates image views of B₁⁺ brain maps of one axial slice calculated from 2D DAM, 2D SDAM, multislice (MS) SDAM, MS DREAM, 3D AFI, and the 3D RF-prepared three-parameter method with complex-subtraction using the double- and single-sot scans (225 and 450 k-lines per shot) with 3D FLASH acquisition and saturation delays of [0.5, 0.75, 1.0, 2.0, 3.0, 5.0, 10.0, 20.0] s.

FIG. 4 illustrates image views of the brain B₁⁺ maps of three subjects from 2D DAM method acquired separately in axial, coronal, and sagittal orientations and the 3D RF-prepared three-parameter method in the corresponding slices with consistent patterns.

FIG. 5 illustrates image views evaluating the 3D RF-prepared B₁ mapping technique in the breast under fixed, adaptive, and SMART shims, respectively.

FIG. 6 illustrates image views breast B₁ maps of three subjects under fixed, adaptive, and SMART shims, respectively, from multislice SDAM method in axial and sagittal orientations and the 3D RF-prepared three-parameter method in the corresponding slices with consistent patterns.

FIG. 7 illustrates image views evaluating the 3D RF-prepared B₁ mapping technique in the abdomen under fixed and adaptive shims, respectively.

FIG. 8 illustrates image views of abdomen B₁⁺ maps of three subjects under fixed and adaptive shims, respectively, by the multislice SDAM method in axial and coronal orientations and the 3D RF-prepared three-parameter method in the corresponding slices with consistent patterns.

FIG. 9 illustrates graphical views of the root mean square error (RMSE) values of each method comparing to 2D DAM or SDAM methods with three orientations for each of the three brains, three shimming conditions for each of three breasts, two shimming conditions for each of the five abdomens.

FIGS. 10A-10D illustrate graphical views associated with acquisitions according to an embodiment of the present invention. FIGS. 10A and 10B illustrate MTF and PSF of FLASH acquisition with low-high profile orders with different Mₚᵣₑₚ values, respectively.

FIG. 11 illustrates image views of the brain B₁⁺ maps of one axial slice calculated from 2D DAM, 2D SDAM, multi-slice (MS) SDAM, MS DREAM, 3D AFI, and the 3D RF-prepared three-parameter method with complex-subtraction using [25, 50, 100, 225, 450] k-lines per shot with 3D FLASH acquisition and saturation delays of [0.5, 1.0, 3.0] s.

FIG. 12 illustrates image views of the breast B₁⁺ maps of one axial slice under fixed, adaptive, and SMART shims, respectively, calculated from 2D DAM, 2D SDAM, multi-slice (MS) SDAM, MS DREAM, 3D AFI, and the 3D RF-prepared three-parameter method with complex-subtraction.

FIG. 13 illustrates image views of arrays of the B₁⁺ scale maps of one axial slice of another subject's abdomen calculated from five methods (DAM was not performed) at two different shimming conditions.

FIG. 14 illustrates graphical views of the error mean values of each method comparing to 2D DAM or SDAM methods with three orientations for each of the three brains, three shimming conditions for each of three breasts, two shimming conditions for each of the five abdomens.

FIG. 15 illustrates graphical views of error standard deviation (SD) values of each method comparing to 2D DAM or SDAM methods with three orientations for each of the three brains, three shimming conditions for each of three breasts, two shimming conditions for each of the five abdomens.

FIG. 16 illustrates graphical views of concordance correlation coefficient (CCC) values of each method comparing to 2D DAM or SDAM methods with three orientations for each of the three brains, three shimming conditions for each of three breasts, two shimming conditions for each of the five abdomens.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains, having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

Gradient echo (GRE) imaging is often chosen as an acquisition scheme for quantitative MRI with magnetization-prepared contrast modules as GRE images do not have severe distortion or signal drop in areas with severe B₀ field inhomogeneity. The point spread function analysis of rapid GRE acquisition shows that the prepared longitudinal magnetization before the GRE acquisition and the image signal obeys a linear (not proportional) relationship. The intercept of the linear function causes a quantification bias for magnetization-prepared GRE imaging due to the k-space filtering effect induced by the T₁ or T₂ relaxation during the long echo train. This effect has traditionally limited the GRE acquisition to be of short duration, such as in two-dimension (2D) or with fast T₁ or T₂ relaxation.

The present invention is a method for quantitative MRI. The method includes using a modality such as 3D FLASH or balanced steady-state free precession (bSSFP). A straightforward approach to obtain many MR contrast with 3D volumetric coverage is to apply a magnetization-preparation module followed by a rapid acquisition module, which could be EPI, fast spin echo (FSE), gradient and spin echo (GRASE), FLASH, and balanced SSFP (bSSFP), etc. It is desirable to choose a 3D acquisition scheme with high SNR efficiency while filling as many phase-encoding (PE) k-lines as possible right after each magnetization-preparation module. The signal decay or recovery due to T_{2*}, T₂ or T₁ relaxation through an extended acquisition duration imposes a k-space filtering effect, which depends on the acquisition scheme, as well as the low-high (centric, center k-lines first) or linear (sequential) profile order being used.

This k-space filtering effect is described as a real modulation transfer function (MTF) in the k-space and a symmetric point spread function (PSF) in the image space as a Fourier transform pair. The PSF has been analytically derived for T₂ exponential decay during a long echo train for FSE type of sequences (including GRASE) with 180° refocusing pulses. The amplitude of the signal can be represented by the maximum magnitude of a PSF function. When considering both the amplitude-loss effect induced by the T₂ decay and the SNR gain from the long acquisition duration based on MR sampling theory, the maximum SNR per unit time can be obtained when the echo train duration is about 1.2T₂ for a Cartesian PE direction (for example, any phase encoding direction in Cartesian scans, partition dimension in stack-of-radial or stack-of-spiral scans).

The blurring of the image can be characterized in terms of FWHM of the PSF function, which describes the broadness of the peak at half of the maximum magnitude. FWHM increases with longer echo train duration following a quadratic function. Similar analysis has been conducted for 1D, 2D, and 3D radial k-space trajectories in ultrashort echo-time imaging and could be expanded to the T₂* decay effect through PE lines of EPI readout. These analyses indicate that EPI or FSE/GRASE sequences suffer strong signal loss and image blurring with a prolonged acquisition window, with its optimal duration on the order of T₂* or T₂, respectively.

Rapid gradient-echo (GRE) imaging such as FLASH and bSSFP are alternative acquisition strategies that are often selected for steady-state imaging. FLASH applies gradient- and/or RF-spoiling at the end of each short TR to eliminate transverse magnetization, yielding pure T₁ weighting. bSSFP does not apply spoiling gradients between alternating excitation pulses and its steady-state signal is known to exhibit a T₂/T₁ contrast. GRE sequences are typically short in each k-line readout and thus do not have severe distortion or signal drop in areas with severe B0 field inhomogeneity. Magnetization-prepared methods always acquire transient magnetization before the steady-state is reached, with low-high profile order typically employed. FLASH, or called as spoiled gradient echo (SPGR), turbo FLASH (TFL), turbo field echo (TFE) by different vendors, has been incorporated for magnetization-prepared sequences to generate different contrast, e.g., T₁, T₂, diffusion, perfusion, functional MRI, MT, CEST, MRA, and water-fat separation. bSSFP has also been applied immediately after different contrast preparations, e.g., T₂, diffusion, T₁ rho, and MRA. It is understood that the signal evolution of the GRE acquisitions influences the obtained image contrast in the PE direction. The k-space filtering effects with regard to MTF have been analytically described for the transient phase of FLASH during the late 1990's and of bSSFP in the early 2000's, respectively. Their MTFs depend on TR, flip angle (FA), the number of PE steps per shot (N), and T₁ (additional T₂ factor for bSSFP). As observed in these earlier studies about the MTF transitioning from the prepared magnetization (Mprep) towards the steady-state (Mss), the signal acquired at the later excitation steps is a summation of one Mprep-proportional term and one Mprep-independent term. The postulation was made subsequently that the image contrast of FLASH would also carry such a linear (not proportional) relationship with Mprep, and the Mprep-independent term as an intercept should be either fitted or corrected for quantitative parameter mapping.

However, as no detailed analysis of the PSF was ever given, the quantification bias was gradually overlooked by many following works employing transient 3D GRE acquisitions, and blurring is still commonly mentioned in numerous papers as an adverse effect of 3D GRE sequences without specification of its relative degree. Choosing an optimal 3D acquisition scheme for a wide range of applications with contrast preparation will benefit from a full understanding of its PSF. Here we aim to provide rigorous analytical derivations of PSFs of both FLASH and bSSFP acquisitions based on general sequence parameters and relaxation values, and characterize their relative contrast, quantification bias and blurring effect. Note that, for GRE, relative contrast instead of relative signal is investigated for PSF analysis due to its signal eventually approaching a steady-state regardless of Mprep. Furthermore, the properties in terms of contrast loss and image blurring will be compared among FLASH, bSSFP and FSE based acquisitions with strategies discussed for optimizing the respective sequence parameters.

For more accurate quantitation based on magnetization-prepared GRE scans using a long GRE acquisition, such as the one employed in three-dimension (3D), a novel imaging method to capture this bias and a following analysis approach to correct such bias are described, herein. The method of the present invention includes performing a quantitative scan. The quantitative scan is followed immediately by a long GRE readout and complex image format. The analysis approach is to treat this saturated image with positive or negative polarities and correct the bias from other magnetization-prepared images with either complex-subtraction or model-fitting.

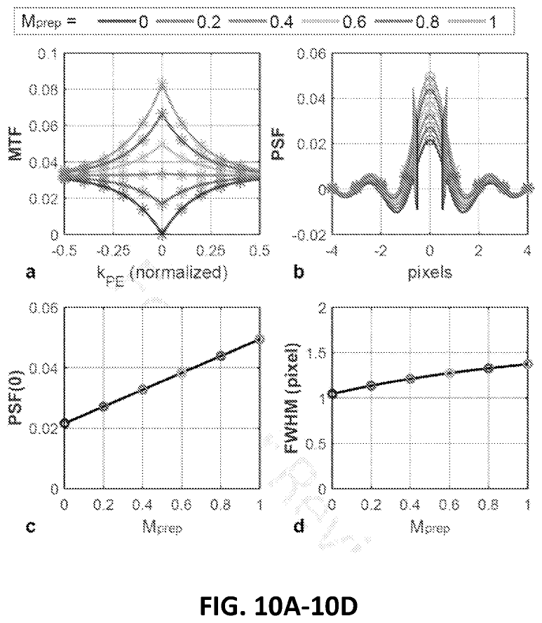

FIGS. 10A-10D illustrate graphical views associated with acquisitions according to an embodiment of the present invention. FIGS. 10A and 10B illustrate MTF and PSF of FLASH acquisition with low-high profile orders with different Mₚᵣₑₚ values, respectively. The analytical results (solid lines) align well with the numerically computed and discretely sampled values (stars) generated via Bloch equation simulations. When Mₚᵣₑₚ=0, the graphical view of FIG. 10A shows MTF of FLASH converge towards the steady-state signals and the graphical view of FIG. 10B shows that the center of their PSFs are nonzero. FWHM (labeled with paired vertical bars) shows little variations with different Mₚᵣₑₚ values. The graphical view of FIG. 10C illustrates the analytically derived linear functions of PSF(0) and the graphical view of FIG. 10D the numerically solved FWHM (y-axis) vs Mₚᵣₑₚ (x-axis) are displayed with the solid lines, which fit closely with the PSF(0) and FWHM for exemplary Mₚᵣₑₚ values (colored circles) for FLASH.

An aspect of the present invention is directed to an RF-prepared approach that employs a 3D FLASH acquisition with a long echo train duration for ultrafast B₁ mapping. A third scan is added with the saturation module immediately followed by the same readout, in order to record the bias from the k-space filtering effect induced by the T₁ relaxation during the long echo train. This bias needs to be subtracted from the first two images. To speed up the imaging time to seconds, the dual refocusing echo acquisition mode (DREAM) method applies a STEAM preparation including two RF pulses followed by a 2D FLASH acquisition to obtain B₁ information in a single shot. A more straightforward scheme is to apply a simple preparation module with only a single RF pulse for FA-encoding before the 2D FLASH or 2D fast spin echo acquisition (termed as RF-prepared method).

An RF-prepared method acquires two scans in a relatively short time, which starts with an imaging module and a fixed saturation delay to remove the T₁ dependence as applied in SDAM, and precedes a 2D FLASH readout with the RF preparation module (for FA-encoding) in one scan and without this module (for normalizing) in another scan. By having separate preparation, the acquisition can be designed more flexibly.

In a particular implementation, the imaging method is to perform a separate scan with a B₁-insensitive saturation module immediately followed by the same long GRE readout and complex image format should be recorded. As stated above, the analysis approach is to treat this saturated image with positive or negative polarities and correct the bias from other magnetization-prepared images with either complex-subtraction or model-fitting. The B₁-insensitive saturation module is used herein as an example to illustrate the invention. However, it should be noted that this example of the B₁-insensitive saturation module is included only by way of example and should not be considered limiting.

In the B₁-insensitive saturation module implementation, one of the RF-prepared methods acquires two scans in a relatively short time, which starts with a B₁-insensitive saturation module and a fixed saturation delay to remove the T₁ dependence as applied in SDAM, and precedes a 2D FLASH readout with the RF preparation module (for FA-encoding) in one scan and without this module (for normalizing) in another scan. Thus, this last method eliminates any potential mismatch of RF profiles of two FAs in the presence of chemical shift (such as fat) or B₀ inhomogeneity potentially faced by DAM or SDAM methods. Furthermore, by having separate preparation, the acquisition can be designed more flexibly.

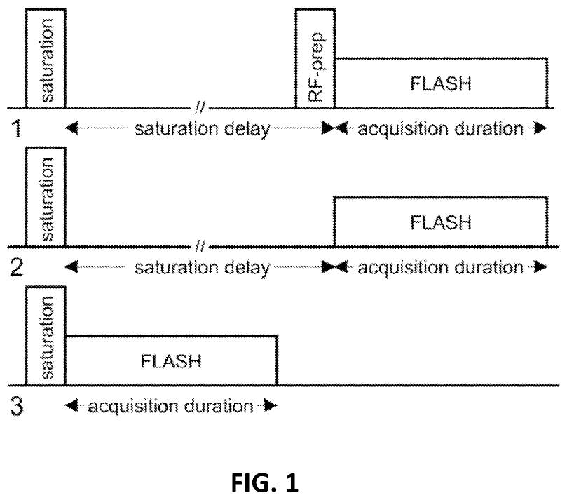

FIG. 1 illustrates a schematic of a proposed image acquisition protocol of the present invention for the exemplary B₁-insensitive saturation module implementation. FIG. 1 illustrates a pulse sequence of the 3D RF-prepared B₁ mapping technique including three separate scans required for the proposed three-parameter method. A B₁-insensitive saturation module followed by a fixed saturation delay is used to generate a consistent magnetization state for the first and the second scans. The same 3D FLASH acquisition with low-high profile ordering is applied for all three scans. In the first scan, a RF-prep module with a short hard pulse (FA=α) is followed by FLASH readout to generate a FA-encoded image. In the second scan, a normalizing image without applying the RF-prep module is acquired; In the third scan, the FLASH acquisition is applied immediately after the saturation module in order to obtain the imaging bias induced by the T₁ relaxation during the long readout.

The first two scans for FA-encoding and normalizing are conducted as in a typical RF-prepared method. The FA can be derived from the ratio of the longitudinal magnetization after an RF-prep pulse with FA=α, MFA, and the normalizing magnetization without this RF-prep pulse, Mₙₒᵣₘ:

$$\frac{{M}_{F A}}{{M}_{n o r m}} = \frac{{M}_{n o r m} cos ( α )}{{M}_{n o r m}} = cos ( α ) \qquad [ 1 ]$$

As done in SDAM, a B₁-insensitive saturation module is used to establish M_{ref} after saturation recovery during a fixed delay. With a two-parameter method, it is assumed that the acquired signal via a FLASH readout (S_{FA} and Sₙₒᵣₘ) are proportional to the prepared magnetizations, S_{FA}=C·MFA and Sₙₒᵣₘ=C·Mₙₒᵣₘ, where C is a constant determined by voxel sizes and coil specifications. Note that the signal evolution due to T₁ recovery through the FLASH acquisition leads to a k-space filtering effect (called partial saturation effect in the original paper), which is not proportional to the prepared magnetization. To keep this effect ignorable, the two-parameter method is limited to 2D acquisition with a short readout duration.

Based on analysis of the point spread function (PSF) for FLASH acquisition, the signal modulation from this effect could be mainly characterized by the peak magnitude of the main lobe of the PSF(r), designated as PSF(0), where the spatial location r=0 is the center of the spatial function. The signal intensity of a FLASH acquisition is proportional to PSF(0), which is found to bear a linear relationship with the initial longitudinal magnetization, Mₚᵣₑₚ:

$$S = C \cdot PS F ( 0 ) = C \cdot ( a \cdot Mprep + b ) \qquad [ 2 ]$$

where a is a slope and b is the intercept.

Thus, for the first two scans with arbitrarily long readout durations,

$${S}_{n o r m} = C \cdot ( a \cdot {M}_{n o r m} + b ) \qquad [ 3 ]$$

$${S}_{F A} = C \cdot ( a \cdot {M}_{n o r m} \cdot cos ( α ) + b ) \qquad [ 4 ]$$

As a result, the FA calculated with the traditional two-parameter method would lead to biased estimation.

In this work, the intercept b is obtained via a third image, similar to the normalizing scan but without the saturation delay. Hence the signal is acquired immediately after the longitudinal magnetization is fully saturated to 0 by the saturation module:

$${S}_{s a t} = C \cdot ( a \cdot 0 + b ) = C \cdot b \qquad [ 5 ]$$

Using a three-parameter method, the bias in FA estimation can thus be corrected:

$$\frac{{S}_{FA} {S}_{s a t}}{{S}_{norm} - {S}_{s a t}} = \frac{C \cdot ( α \cdot {M}_{n o r m} \cdot cos ( α ) + b ) - C \cdot b}{C \cdot ( α \cdot {M}_{n o r m} + b ) - C \cdot b} = cos ( α ) \qquad [ 6 ]$$

The relationship of the actual FA (α) and the nominal FA (α^{norm}) is usually described with a B₁⁺

scale factor κ (with 100% as consistent between the two):

$$κ = \frac{a}{{a}^{n o m}} \times 100 \% = \frac{arc cos ( \frac{{S}_{F A} - {S}_{s a t}}{{S}_{norm} - {S}_{s a t}} )}{{a}^{n o m}} \times 100 \% \qquad [ 7 ]$$

It is important to note that, although the formulas above are based on the main lobe of the PSF, which implies that intercept b is a positive value, Sₛₐₜ signal may be affected by the side lobes of PSF of surrounding pixels as well, which could have negative b values (FIGS. 10A-10D). Thus, subtractions between S_{FA}, Sₙₒᵣₘ and Sₛₐₜ should consider opposite polarities and complex-subtraction is preferred in this case. Using complex values also avoids errors in B₁⁺ estimation when α>90°.

In vivo datasets of three brains, three breasts and five abdomens were obtained from six volunteers (4 females, 2 males; 25-45 years old). The study was approved by the Johns Hopkins School of Medicine Institutional Review Board and all subjects provided informed consent. Experiments were conducted on a 3T scanner (Ingenia; Philips Medical Systems, Best, The Netherlands), with the body coil (maximum amplitude 13.5 μT) equipped with a dual-source parallel RF excitation system. The maximum strength of the gradient coil was 40 mT/m and the maximum slew rate was 200 mT/m/ms. A 32-channel head coil, a 16-channel bilateral breast coil and a 32-channel chest coil were used for signal reception in brain, breast, and abdomen scans, respectively.

For the three-parameter method (FIG. 1), the RF-prep module for the FA-encoding scan utilized a 0.29 ms non-selective hard pulse with αnom=60° immediately followed by spoiler gradients to dephase any residual transversal magnetization. All three scans applied a common saturation module, which used a B₁-insensitive WET pre-pulse consisting of four pulses with different FAs: 72°, 92°,126°, and 193° interspersed with crusher gradients to null transversal magnetization.

The FLASH acquisition had a low-high profile ordering with the center of the k-space acquired at the beginning of the readout, and the specific parameters for brain scans were: TR/TE=3.4/2.3 ms, FOV=220×220×120 mm³, imaging matrix=56×56×30, acquisition and reconstruction resolution=4×4×4 mm³, readout bandwidth (BW)=2975 Hz/pixel, compressed-sensing (CS) acceleration factor=3. To investigate the effect of acquisition duration and saturation delay, the numbers of k-lines per shot were varied, [25, 50, 100, 225, 450], to have a different number of shots, [24, 12, 6, 2, 1]. With TR of 3.4 ms, the subsequent acquisition durations per shot were [86, 172, 343, 771, 1542] ms. For each acquisition scheme, corresponding acquisition FAs of [18°, 13°, 9°, 6°, 5°] were chosen for optimal signal contrast per unit time based on the PSF analysis for FLASH using a typical gray matter T₁ (1400 ms). Different saturation delays of [0.5, 1.0, 3.0] s were evaluated for all these scans while additional [0.75, 2.0, 5.0, 10.0, 20.0] s were added for the double- and single-sot scans. For the proposed three-parameter method with a single-shot acquisition (450 k-lines per shot, 1.54 s acquisition duration) and 2.0 s saturation delay (no saturation delay used in the third image), the total scan time was only 8.6 s.

Five other existing B₁ mapping methods with matched FOV, resolution/slice thickness, and CS factor were implemented to compare with the 3D RF-prepared method in the brain.
1) Single-slice DAM with 60°/120° excitation FA by 0.58 ms non-selective hard pulses and single-shot fast-spin-echo acquisition (echo space of 3.7 ms, slice-selective refocusing FA=180°), low-high profile ordering, readout BW=1691 Hz/pixel, TR=20 s, total scan duration 40 s. For comparison with other methods with either 3D or multislice coverage, three separate scans were performed in three orthogonal orientations at the center of the slabs.
2) Single-slice SDAM with the same acquisition as used in DAM, the same saturation module chosen in the RF-prepared protocol with a saturation delay of 0.5 s, TR=0.58 s, total scan duration=1.2 s. Three separate scans were performed in three orthogonal orientations.
3) Multi-slice SDAM with 2D SDAM repeated for 30 slices, readout BW=1583 Hz/pixel, TR=17 s, total scan duration=34 s.
4) Multi slice DREAM with single-shot 2D FLASH acquisition of 30 slices, low-high profile ordering, with TR/TESTE/TEFID=4.5/1.7/2.3 ms (stimulated echo first), STEAM/FLASH FA=60°/15° and their slice thickness ratio=2, readout BW=3382 Hz/pixel, saturation effects mitigated by scanning odd slices first and then even slices with equal temporal spacings of 0.5 s and the acquisition time between neighboring slices=8.5 s, total scan duration=17 s.
5) AFI with 3D FLASH acquisition of 30 slices, TR1/TR2/TE=20/100/4.6 ms, FA=60° readout BW=435 Hz/pixel, with adequate RF and gradient spoiling, total scan duration=148 s.

For breast and abdomen scans, the specific acquisition parameters of the RF-prepared protocol were: FOV=350×350×180 mm³, imaging matrix=58×58×30, acquisition and reconstruction resolution=6×6×6 mm³, readout bandwidth=2854 Hz/pixel, CS acceleration factor=3. The single-shot acquisition (450 k-lines per shot) and 2.0 s saturation delay was chosen from the brain scan results for its efficiency (total scan duration kept 8.6 s). Four or five other B₁ mapping methods were also evaluated respectively with matched acquisition parameters. Only axial scans were performed for single-slice DAM and SDAM methods.

All abdomen scans were acquired during one breath-hold. The single-slice DAM method was not applied for the abdomen scans as its 40 s scan time is not suitable for single breath-hold scans. The multislice SDAM was acquired with 15 slices of 12 mm slice thickness, total scan duration=17 s. the 3D AFI was only acquired for 3 slices of 6 mm slice thickness, total scan duration=15 s.

B₁ shimming with standard single-source RF excitation (“Fixed”) was used for all three organs. For breast and abdomen scans, a dual-source B₁ shimming (“Adaptive”) was also performed. A vendor-provided B₁ shimming method based on geometries of individual breasts (“SMART”) was also applied for breast scans.

For the 3D RF-prepared method, both magnitude and complex images after vendor-provided CS reconstruction were recorded. B₁⁺ scale maps of the proposed 3D RF-prepared three-parameter method were calculated with Eq. [7] with both magnitude- and complex-subtractions between the FA-encoded, the normalizing images and the saturated image pixel by pixel. The two parameter results (without subtracting the saturated image) were also calculated for comparison. Respective B₁ maps based on other B₁ methods were computed as well. No image filtering was performed for any B₁ maps. A mask for each B₁ map was manually drawn from respective raw images to remove bones and only include brain, breast, and abdomen tissues. Within the mask area, the B₁ maps were quantitatively compared with the DAM (SDAM for abdomen scans) results using four metrics: 1) root mean square error (RMSE), 2) error mean, 3) error standard deviation (SD), 4) concordance correlation coefficients (CCC).

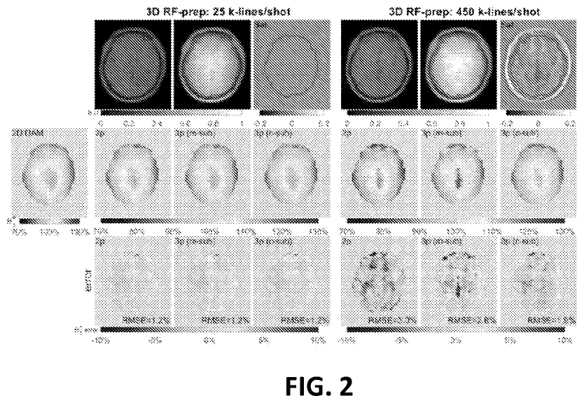

The FA-encoded, normalizing and saturated images of an axial slice of a subject's brain, using 25 and 450 k-lines per shot for 3D FLASH acquisition and a saturation delay of 3.0 s, were shown in the first row of FIG. 2. The calculated B₁ maps from the 2D DAM method, the 3D RF-prepared two-parameter method, and the proposed three-parameter method using both magnitude- and complex-subtractions are displayed in the second row of FIG. 2. The third row exhibits the pixel-wise difference between the B₁ maps derived from 3D RF-prepared methods and 2D DAM method (RF-prepared-DAM). With only 25 k-lines per shot, the signal in the saturated image is rather small and the two- and three-parameter methods yielded similarly small errors (RMSE=1.2%). When increasing to 450 k-lines per shot, the signal in the saturated image was higher and the two-parameter method generated higher errors (RMSE=3.0%), which was largely corrected via the three-parameter method with complex-subtraction (RMSE=1.9%).

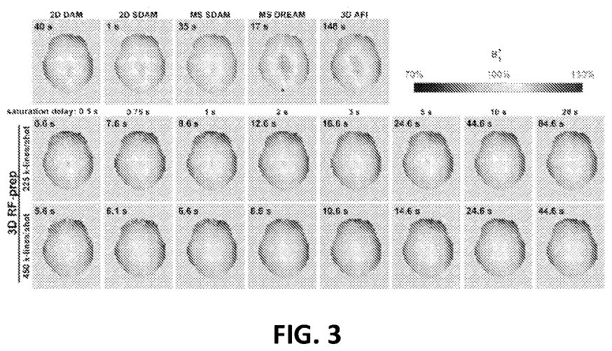

FIG. 3 illustrates image views of B₁⁺ brain maps of one axial slice calculated from 2D DAM, 2D SDAM, multislice (MS) SDAM, MS DREAM, 3D AFI, and the 3D RF-prepared three-parameter method with complex-subtraction using the double- and single-sot scans (225 and 450 k-lines per shot) with 3D FLASH acquisition and saturation delays of [0.5, 0.75, 1.0, 2.0, 3.0, 5.0, 10.0, 20.0] s. The total scan time for each method is labeled at each top left corner. It can be observed that the protocol with single-shot acquisition (450 k-lines per shot) and 2.0 s saturation delay produced sufficient quality in only 8.6 s.

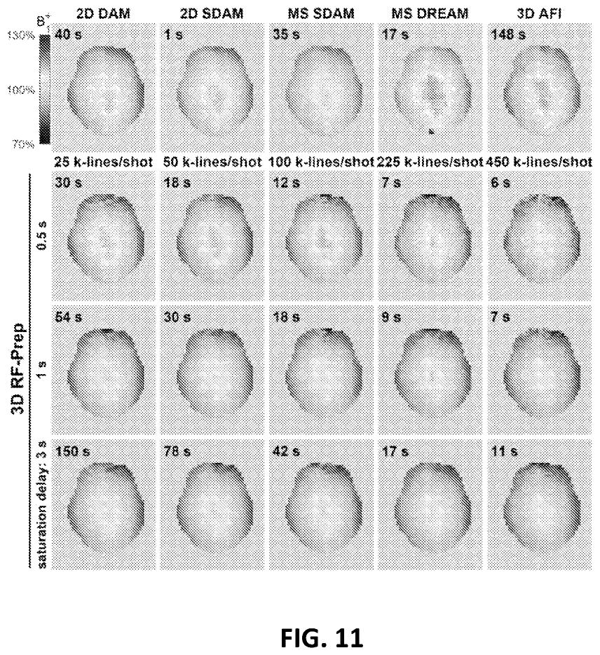

FIG. 3 and FIG. 11 illustrate image views of arrays the B₁⁺ scale maps of one axial slice of another subject's brain calculated from single-slice DAM, single-slice SDAM, multislice SDAM, DREAM, 3D AFI, and the 3D RF-prepared three-parameter method with complex subtraction using different numbers of k-lines per shot with 3D FLASH acquisition and various saturation delays. All the results of the three-parameter method produced consistent B₁⁺ scale distributions resembling five other methods (first row). The protocols with shorter saturation delays (less than 1 s) yielded lower SNR as expected. The single-shot acquisition (450 k-lines per shot) and 2.0 s saturation delay produced sufficient quality in only 8.6 s, thus were chosen as the recommended parameters and are the default setting for the three-parameter method mentioned below when not specified otherwise.

FIG. 11 illustrates image views of the brain B₁⁺ maps of one axial slice calculated from 2D DAM, 2D SDAM, multi-slice (MS) SDAM, MS DREAM, 3D AFI, and the 3D RF-prepared three-parameter method with complex-subtraction using [25, 50, 100, 225, 450] k-lines per shot with 3D FLASH acquisition and saturation delays of [0.5, 1.0, 3.0] s. The total scan time for each method is labeled at each top left corner.

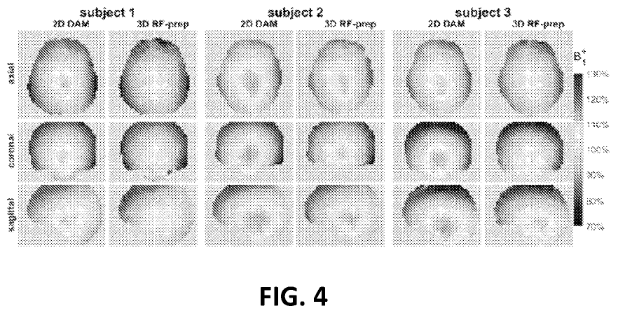

B₁⁺ scale maps of three different brains from the 2D DAM method acquired separately in axial, coronal, and sagittal orientations show consistent patterns with the results from the corresponding slices from the 3D RF-prepared three-parameter method in FIG. 4, with B₁⁺ scales highest in the center of the cranium (~120%) reducing to lowest at the peripheral and superior of the brain (~80%).

FIG. 4 illustrates image views of the brain B₁⁺ maps of three subjects from 2D DAM method acquired separately in axial, coronal, and sagittal orientations and the 3D RF-prepared three-parameter method in the corresponding slices with consistent patterns.

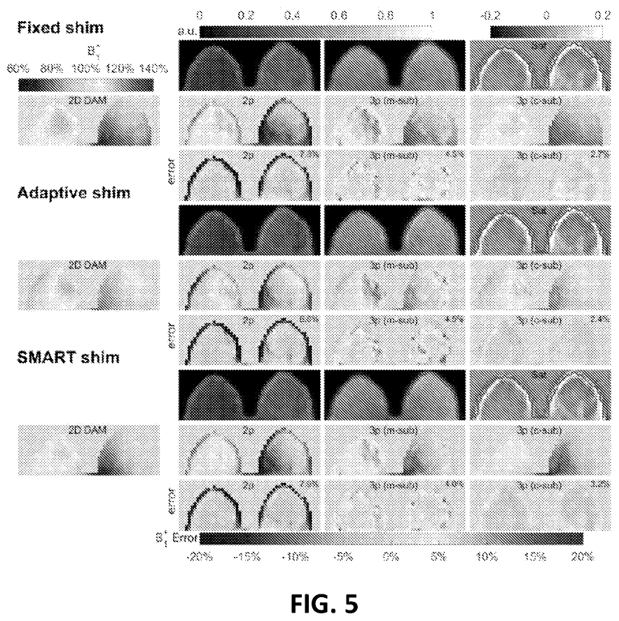

FIG. 5 illustrates image views evaluating the 3D RF-prepared B₁ mapping technique in the breast under fixed, adaptive, and SMART shims, respectively. For each shim condition, the top images show the FA-encoded, the normalizing, and the saturated images, with the tissue masks labeled in solid lines; the middle images show the calculated B₁⁺ maps using the 2D DAM method, the 3D RF-prepared two-parameter method, and the three-parameter method with both magnitude- and complex-subtractions; and the bottom images show their corresponding error maps (RF-prepared-DAM) with the averaged RMSE (root mean square error) indicated at the top right corners. FA: FA-encoded; norm: normalizing; sat: saturated; 2p: two-parameter method; 3p: three-parameter method; m-sub: magnitude-subtraction; c-sub: complex-subtraction

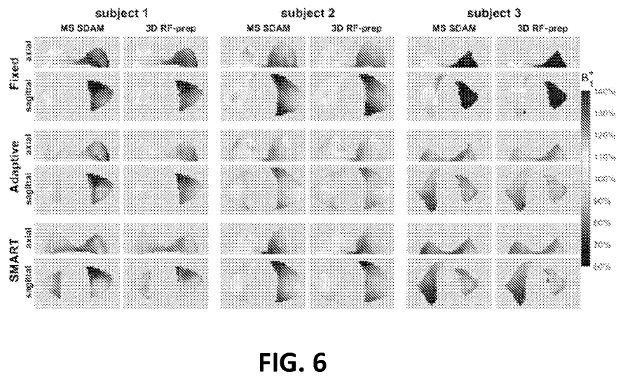

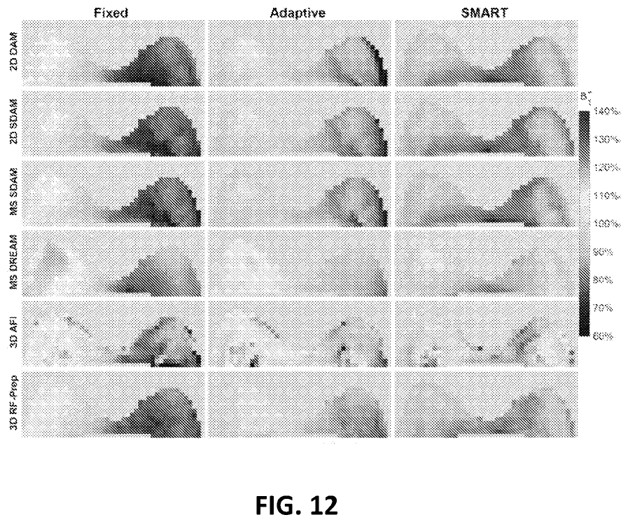

FIG. 5 shows the results of an axial slice of a subject's breast with the calculated B₁⁺ scale maps from the 2D DAM method, the 3D RF-prepared two-parameter method and the proposed three-parameter method using both magnitude- and complex-subtractions, and their respective difference maps, under fixed, adaptive, and SMART shims respectively. FIG. 12 illustrates image views of arrays of the B₁⁺ scale maps of one axial slice of another subject's breast calculated from all six methods at three different shimming conditions. B₁⁺ scale maps of three different breast at three different shimming conditions from multislice SDAM method in axial and sagittal orientations show consistent patterns with the results from the corresponding slices of the 3D three-parameter method in FIG. 6, with the differences of B₁⁺ scales between left and right breasts reduced from fixed shims to adaptive and SMART shims.

FIG. 12 illustrates image views of the breast B₁⁺ maps of one axial slice under fixed, adaptive, and SMART shims, respectively, calculated from 2D DAM, 2D SDAM, multi-slice (MS) SDAM, MS DREAM, 3D AFI, and the 3D RF-prepared three-parameter method with complex-subtraction.

FIG. 6 illustrates image views breast B₁ maps of three subjects under fixed, adaptive, and SMART shims, respectively, from multislice SDAM method in axial and sagittal orientations and the 3D RF-prepared three-parameter method in the corresponding slices with consistent patterns.

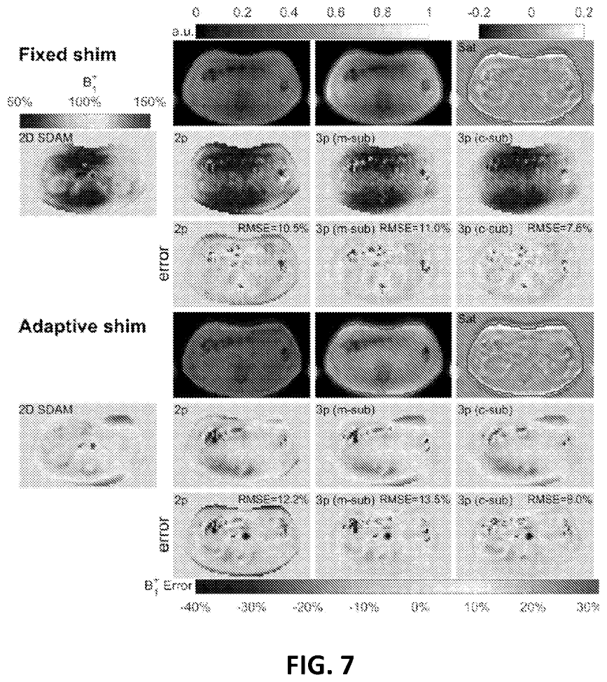

FIG. 7 illustrates image views evaluating the 3D RF-prepared B₁ mapping technique in the abdomen under fixed and adaptive shims, respectively. For each shim condition, the top images show the FA-encoded, the normalizing, and the saturated images, with the tissue masks labeled in red solid lines; the middle images show the calculated B₁⁺ maps using the 2D DAM method, the 3D RF-prepared two-parameter method, and the three-parameter method with both magnitude- and complex-subtractions; the middle images show their corresponding error maps (RF-prepared-DAM) with the averaged RMSE (root mean square error) indicated at the top right corners. FA: FA-encoded; norm: normalizing; sat: saturated; 2p: two-parameter method; 3p: three-parameter method; m-sub: magnitude-subtraction; c-sub: complex-subtraction.

FIG. 7 further shows the results of an axial slice of a subject's abdomen with the calculated B₁⁺ scale maps from the 2D SDAM method, the 3D RF-prepared two-parameter method and the proposed three-parameter method using both magnitude- and complex-subtractions, and their respective difference maps, under fixed and adaptive shims respectively.

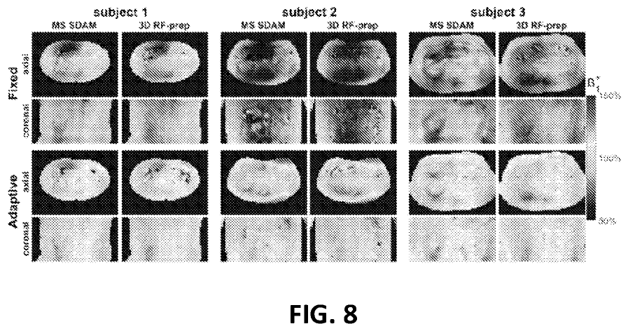

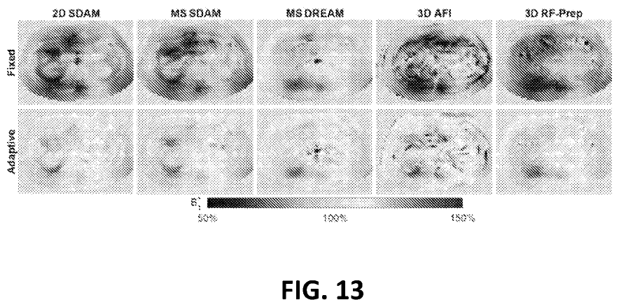

FIG. 13 illustrates image views of arrays of the B₁⁺ scale maps of one axial slice of another subject's abdomen calculated from five methods (DAM was not performed) at two different shimming conditions. Abdominal B₁⁺ maps of three subjects at two different shimming conditions from multislice SDAM method in axial and coronal orientations show consistent patterns with the results from the corresponding slices of the 3D three-parameter method in FIG. 8, with the large B₁ shading effect under fixed shims mitigated under adaptive shims. Further with respect to FIG. 13, the images illustrate the abdomen B₁⁺ maps of one axial slice under fixed, adaptive, and SMART shims, respectively, calculated from 2D SDAM, multi-slice (MS) SDAM, MS DREAM, 3D AFI, and the 3D RF-prepared three-parameter method with complex-subtraction.

FIG. 8 illustrates image views of abdomen B₁⁺ maps of three subjects under fixed and adaptive shims, respectively, by the multislice SDAM method in axial and coronal orientations and the 3D RF-prepared three-parameter method in the corresponding slices with consistent patterns.

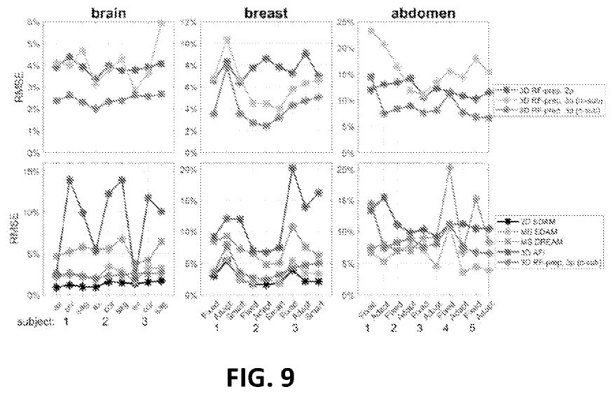

FIG. 9 illustrates graphical views of the root mean square error (RMSE) values of each method comparing to 2D DAM or SDAM methods with three orientations for each of the three brains, three shimming conditions for each of three breasts, two shimming conditions for each of the five abdomens. Across three organs, the top graphical views show RMSE of the 3D RF-prepared three-parameter method with complex-subtraction are consistently lower than the results of two-parameter method or three-parameter method with magnitude-subtraction; the bottom graphical views show RMSE of the 3D RF-prepared three-parameter method with complex subtraction are consistently lower than the results of 3D AFI and are close to the results of 2D or MS SDAM for all three organs, and are consistently lower than the results of MS DREAM in the brain and breast and are lower or close to in the abdomen.

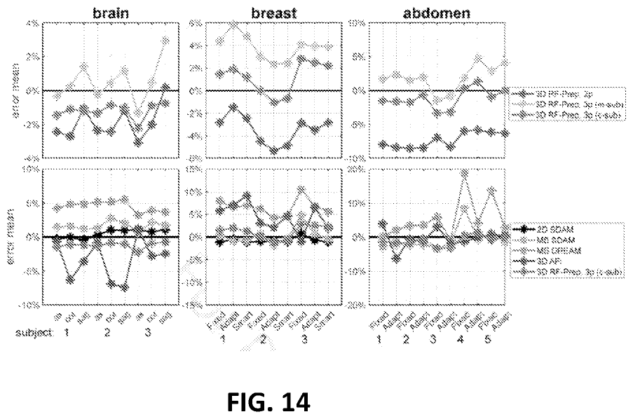

When combining comparison results from all three organs of all the subjects, including three orientations for each of the three brains, three shimming conditions for each of three breasts, two shimming conditions for each of the five abdomens, the RMSE, error mean, error SD, and CCC values of each B₁ mapping method comparing to 2D DAM or SDAM methods for each organ are plotted in FIG. 9 and FIGS. 14, 15, and 16, respectively.

FIG. 14 illustrates graphical views of the error mean values of each method comparing to 2D DAM or SDAM methods with three orientations for each of the three brains, three shimming conditions for each of three breasts, two shimming conditions for each of the five abdomens. Across three organs, the top graphical views illustrate error mean of the 3D RF-prepared three-parameter method with complex-subtraction are consistently lower than the results of two-parameter method or three-parameter method with magnitude-subtraction; the bottom graphical views show error mean of the 3D RF-prepared three-parameter method with complex-subtraction are consistently lower than the results of MS DREAM and are close to the results of 2D or MS SDAM for all three organs, and are consistently lower than the results of 3D AFI in the brain and breast and are lower or close to in the abdomen.

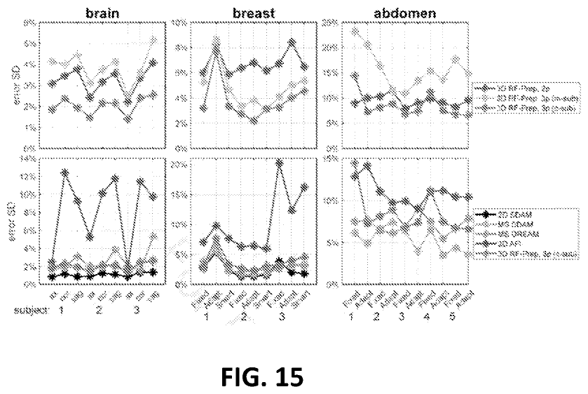

FIG. 15 illustrates graphical views of error standard deviation (SD) values of each method comparing to 2D DAM or SDAM methods with three orientations for each of the three brains, three shimming conditions for each of three breasts, two shimming conditions for each of the five abdomens. Across three organs, (top) error SD of the 3D RF-prepared three-parameter method with complex-subtraction are consistently lower than the results of two-parameter method or three-parameter method with magnitude-subtraction; (bottom) error SD of the 3D RF-prepared three-parameter method with complex-subtraction are consistently lower than the results of 3D AFI and are close to the results of MS DREAM and 2D or MS SDAM for all three organs.

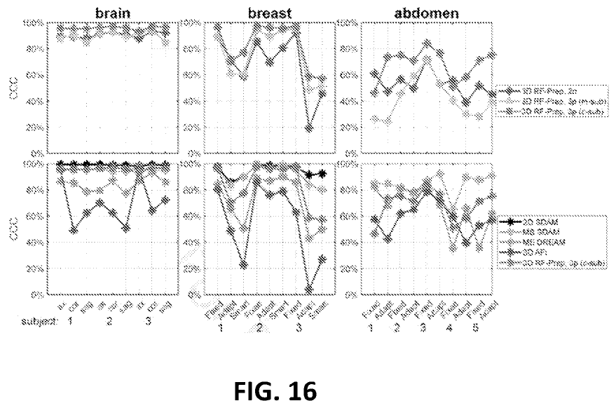

FIG. 16 illustrates graphical views of concordance correlation coefficient (CCC) values of each method comparing to 2D DAM or SDAM methods with three orientations for each of the three brains, three shimming conditions for each of three breasts, two shimming conditions for each of the five abdomens. Across three organs, (top) CCC of the 3D RF-prepared three-parameter method with complex-subtraction are consistently higher than the results of two-parameter method or three-parameter method with magnitude-subtraction; (bottom) CCC of the 3D RF-prepared three-parameter method with complex-subtraction are largely higher than the results of 3D AFI and are close to the results of 2D or MS SDAM for all three organs, and are consistently higher than the results of MS DREAM in the brain and breast and are close to in the abdomen.

Their averaged values across nine or ten different data for each method and each organ are listed in Table 1. Compared to the 3D RF-prepared two-parameter method, the three-parameter method using complex-subtraction yielded the averaged RMSE 38% lower in the brain (2.4% vs. 3.9%), 45% lower in the breast (4.2% vs. 7.7%), and 27% lower in the abdomen (8.7% vs. 11.9%); it also had the averaged error mean values 37% lower in the brain (−1.2% vs. −1.9%), 68% lower in the breast (1.1% vs. −3.4%), and 85% lower in the abdomen (−1.1% vs. −7.3%); its CCC values were 5.9% higher in the brain (95.9% vs. 90.6%), 22% higher in the breast (83.0% vs. 68.1%), and 28% higher in the abdomen (68.2% vs. 53.1%) (Table 1). Note that the three-parameter method using magnitude-subtraction did not show such remarkable improvements (FIG. 9, Table 1). Based on these quantitative metrics, the 3D RF-prepared three-parameter method with complex subtraction delivered consistently lower RMSE, error mean, error SD, and higher CCC values than multislice DREAM and 3D AFI, and were close to the results of 2D or multislice SDAM (FIG. 9, Table 1).

TABLE 1
The root mean square error (RMSE), error mean, error standard deviation (SD),
and concordance correlation coefficient (CCC) values of each B₁ mapping
method comparing to 2D DAM or SDAM methods averaged across three orientations
for each of the three brains, three shimming conditions for each of three
breasts, two shimming conditions for each of the five abdomens.

| | | | |
|---|---|---|---|
| RMSE | Error Mean | Error SD | CCC |

BRAIN

| 2D SDAM | | 1.3 ± 0.3 | 0.5 ± 0.6 | 1.1 ± 0.2 | 98.9 ± 0.4 |
|---|---|---|---|---|---|
| MS SDAM | | 2.6 ± 0.7 | 1.6 ± 0.6 | 2.0 ± 0.5 | 95.5 ± 1.1 |
| MS DREAM | | 5.3 ± 1.0 | 4.5 ± 0.8 | 2.7 ± 1.2 | 84.5 ± 5.1 |
| 3D AFI | | 9.1 ± 4.6 | −3.3 ± 3.0 | 8.2 ± 4.1 | 69.1 ± 16.9 |
| 3D RF- | 2p | 3.9 ± 0.3 | −1.9 ± 1.0 | 3.2 ± 0.6 | 90.6 ± 2.1 |
| PREP | 3p (m-sub) | 4.1 ± 0.9 | 0.6 ± 1.2 | 3.9 ± 0.8 | 89.9 ± 3.5 |
| | 3p(c-sub) | 2.4 ± 0.2 | −1.2 ± 0.4 | 2.0 ± 0.4 | 95.9 ± 1.2 |

BREAST

| 2D SDAM | | 2.7 ± 1.2 | −0.7 ± 0.6 | 2.5 ± 1.3 | 94.6 ± 4.8 |
|---|---|---|---|---|---|
| MS SDAM | | 3.4 ± 1.4 | 0.2 ± 1.9 | 2.9 ± 1.3 | 91.5 ± 7.2 |
| MS DREAM | | 7.4 ± 1.9 | 6.6 ± 1.9 | 3.0 ± 1.4 | 71.6 ± 19.3 |
| 3D AFI | | 11.6 ± 4.6 | 4.3 ± 3.1 | 10.2 ± 5.0 | 54.0 ± 29.8 |
| | 2p | 7.7 ± 0.9 | −3.4 ± 1.3 | 6.8 ± 0.9 | 68.1 ± 23.6 |
| | 3p (m-sub) | 6.2 ± 1.9 | 3.9 ± 1.1 | 4.8 ± 1.6 | 76.1 ± 20.1 |
| | 3p (c-sub) | 4.2 ± 1.6 | 1.1 ± 1.4 | 3.8 ± 1.6 | 83.0 ± 17.3 |

ABDOMEN

| MS SDAM | | 6.1 ± 2.2 | −0.8 ± 3.7 | 5.3 ± 1.4 | 84.1 ± 8.2 |
|---|---|---|---|---|---|
| MS DREAM | | 9.8 ± 4.6 | 5.3 ± 6.5 | 7.1 ± 1.0 | 66.0 ± 18.7 |
| 3D AFI | | 11.2 ± 2 | −0.4 ± 2.9 | 11 ± 1.6 | 58.6 ± 12.8 |
| | 2p | 11.9 ± 1.4 | −7.3 ± 1.2 | 9.4 ± 1.1 | 53.1 ± 9.2 |
| | 3p (abs) | 16.0 ± 4.0 | 1.9 ± 1.9 | 15.8 ± 4.1 | 41.8 ± 16.5 |
| | 3p (cpx) | 8.7 ± 2.4 | −1.1 ± 1.5 | 8.5 ± 2.5 | 68.2 ± 12.7 |

2p: two-parameter method; 3p: three-parameter method; m-sub: magnitude-subtraction; c-sub: complex-subtraction.
All values are reported in %.

The proposed three-parameter method enables the existing RF-prepared B₁ mapping technique to be extended to 3D FLASH long readout by correcting the T₁-induced quantification bias. FIG. 2 illustrates image views evaluating the 3D RF-prepared B₁ mapping technique in the brain. The third saturated image added a very short extra time (1.54 s acquisition duration for the single-shot readout), and revealed this non-negligible bias.

FIG. 2 shows evaluation of the 3D RF-prepared B₁ mapping technique in the brain. The top images show the FA-encoded, the normalizing, and the saturated images acquired with 25 and 450 k-lines per shot for 3D FLASH acquisition and a saturation delay of 3.0 s, with the tissue masks labeled in red solid lines. The middle images show the calculated B₁⁺ maps using the 2D DAM method, the RF-prepared two-parameter method, and the three-parameter method with both magnitude- and complex-subtractions. The bottom images show their corresponding error maps (RF-prepared-DAM). Root mean square error (RMSE) of B₁⁺ maps are labeled at the bottom of the corresponding error maps. FA: FA-encoded; norm: normalizing; sat: saturated; 2p: two-parameter method; 3p: three-parameter method; m-sub: magnitude-subtraction; c-sub: complex-subtraction.

B₁ maps were more accurate when subtracting the saturated images from the FA-encoded and normalizing images respectively than without this subtraction (FIGS. 2, 5, 7). With the finding through PSF analysis of a linear (not proportional) relationship between the prepared longitudinal magnetization before the FLASH acquisition and the image signal (Eq. [2]), it became clear that the intercept of the linear function causes the quantification bias for the established two-parameter method. Both a higher number of k-lines per shot and a shorter T₁ value would lead to larger bias, as illustrated in FIG. 10. Another character of the PSF of FLASH is its side lobes oscillating and phase-changing around zero. The tissue signal with a shorter T₁ (i.e. fat, white matter) could turn the surrounding tissue with low intensity due to its longer T₁ (i.e. CSF) to a negative signal. In this case, the magnitude-subtraction would amplify the quantification bias and the complex-subtraction indeed show better performance in different organs (FIGS. 2, 5, 7, 9, 14, 15, and 16, Table 1).

Comprehensive comparisons between the proposed 3D RF-prepared method and five other established B₁ mapping techniques were performed in the brain, breast, and abdomen respectively for either different orientations or different shim conditions. The method of the present invention delivered similar performance as 2D or multislice SDAM, while taking only a quarter of the scan time for multislice SDAM with the same spatial coverage and resolution (8.6 s vs 34 s). Compared to multislice DREAM, the method of the present invention yielded largely improved accuracy (RMSE: 2.4% vs. 5.3% in the brain, 4.2% vs. 7.4% in the breast, 8.7% vs. 9.8% in the abdomen. Table 1) while only taking half of the scan time (8.6 s vs 17 s). Note that the DREAM method requires that fully relaxed longitudinal magnetizations are established prior to the STEAM preparation pulses for each slice. Its accuracy can be affected by the T₁-induced saturation effect, which demands a long waiting time after the last sequence (not counted in the 17 s scan time) and a long temporal delay between neighboring slices.44,45 3D AFI method suffered higher errors (Table 1) and longer scan time (148 s) than all other methods. In addition, ringing artifacts were also observed in AFIderived unfiltered B₁ maps.

Acquiring an additional image with the saturation preparation has also been proposed for improving the quantification model of T₂-prepared myocardial T₂ mapping employing a balanced SSFP acquisition with linear ramp-up pulses and linear profile ordering. In that study, the saturated image was to capture the T₁ relaxation effect by the imaging pulses before acquiring the center of the k-space. In contrast, the present invention is based on the PSF analysis of this k-space filtering effect for largely the low-high profile ordering used in this study (although it also applies to the linear profile ordering). Furthermore, the saturated images were assumed as all positive numbers and only magnitude images were used for fitting the myocardial T₂, while in the current study negative data were observed at some pixels and complex subtraction was shown to be more successful for improving the quantification accuracy (FIGS. 2, 5, 7, 9, 14, 15, and 16, Table 1).

2D DAM with a single-shot fast-spin-echo acquisition was chosen as the reference method. DAM is based on the transverse magnetization after two hard pulses (60° and 120°, 0.58 ms) and 3D RF-prep is based on the longitudinal magnetization after one hard pulse (60°, 0.29 ms). Their different sensitivity to off-resonance or chemical shift such as fat needs to be considered when comparing the two methods. B₀ maps were not acquired in this study, which could provide useful information when evaluating the performance of each B₁ mapping technique. Lastly, the saturation module might be further improved for more robustness to B₀ and B₁ inhomogeneities). Only a modest CS acceleration factor of 3 was used for all B₁ mapping techniques. For the RF prepared approach with a separate 3D acquisition, higher CS acceleration factor or stack-of-spiral GRE readout could be employed to further reduce the acquisition time.

The proposed 3D RF-prepared three-parameter method with complex-subtraction for B₁ mapping delivered consistently higher accuracy in brain, breast, and abdomen than the traditional two-parameter method, the three-parameter method with magnitude-subtraction, the multi-slice DREAM and the 3D AFI, and were close to the results of 2D or multi-slice SDAM, while only taking a fraction of time of existing methods. This technique needs to be evaluated in a larger group of subjects for further evaluation.

The k-space filtering effects with regard to modulation transfer function (MTF) have been analytically described for the transient phase of FLASH during late 1990's. Their MTFs depend on TR, flip angle (FA), the number of PE steps (N), and T₁. As observed in these earlier studies about the MTF transitioning from the prepared magnetization (Mₚᵣₑₚ) toward the steady state (Mₛₛ), the signal acquired at the later excitation steps is a summation of one Mₚᵣₑₚ-proportional term and one Mₚᵣₑₚ-independent term. Postulation was made subsequently that the image contrast of FLASH would also carry such a linear (not proportional) relationship with Mₚᵣₑₚ, and the Mₚᵣₑₚ-independent term as an intercept should be either fitted or corrected for quantitative parameter mapping.

### General Framework—Modulation Transfer Function (MTF) and Point Spread Function (PSF)

Because the k-space filtering effect due to the magnetization relaxation manifest mainly along the slowest PE direction in each echo train (assumed as z direction), the following derivations are limited in 1D along this direction. The MTF applies a multiplication filter onto the original k-space:

$${S}_{k} = {S}_{o r i} ( k ) \cdot MTF ( k ) , \qquad [ 8 ]$$

where k is the k-space location, S(k) is the signal acquired within the k-space, Sₒᵣᵢ(k) is the original signal without magnetization-relaxation induced signal evolution through these k-space locations, which is described as MTF(k). For simplify, the k-space location is normalized to the entire range of the k-space along the assumed z direction [−π/Δz, π/Δz] by dividing 2π/Δz, such that kϵ[−0.5, 0.5].

With N as the number of PE steps acquired in the echo train, and the function M_{xy}(n) as the transverse signal acquired at the n^{th} PE step, the MTF of a linear (LN) and low-high (LH) profile orders are:

$$M T {F}^{L N} ( k ) = {M}_{x y} ( n ( k ) ) = {M}_{x y} ( k + 0.5 ) N + 1 ) \qquad [ 9 ]$$

$$MT {F}^{L H} ( k ) = {M}_{x y} ( 2 \vert k \vert N + 1 ) \qquad [ 10 ]$$

The PSF is the Fourier transform of the MTF:

$$P S F ( z ) = {\int }_{- 0.5}^{0.5} M T F ( k ) {e}^{i 2 π k z} d k , \qquad [ 11 ]$$

where z is the index of image pixels, and i is the imaginary unit. The range of z here is the reciprocal of the k-space resolution (Δk=1/N), so zϵ[−N/2, N/2], The image (I) is a circular convolution of the original image (Iₒᵣᵢ) and the PSF:

$$I ( z ) = Iori ( z ) ⊗ P S F ( z ) \qquad [ 12 ]$$

The convolution with PSF results in two effects on the images as mentioned above, an amplitude-loss effect that can be characterized with the peak magnitude of the main lobe of the PSF, i.e. PSF(0), and a blurring effect that can be characterized with the FWHM of the main lobe of the PSF.

Note that PSF(0) is the integral of the transverse signal acquired at all echoes, which is not affected by the profile order:

$$P S {F}^{L N} ( 0 ) = P S {F}^{L H} ( 0 ) = {\int }_{- 0.5}^{0.5} M T F ( k ) d k = {\int }_{1}^{N} {M}_{x y} ( n ) dn . \qquad [ 13 ]$$

Because the MTF is real, the PSF is an even function. Therefore, the FWHM satisfies abs(PSF(FWHM/2))=PSF(0)/2. As it is difficult to find the analytical solution of the FWHM due to the complexity of the PSF formula, the FWHM is obtained numerically in this work.

### FLASH—MTF and PSF of FLASH

FLASH refers to the acquisition consisting of N excitation pulses with a constant small FA of α, short TR and N PE steps which are centered at the gradient echoes with an echo time of TE. It is assumed that the transverse magnetization before the following FLASH excitation pulses are fully spoiled. Based on the Bloch equation, the longitudinal magnetization at the end of the n^{th} TR is:

$${M}_{z} ( n ) = {M}_{z} ( N - 1 ) ε + {M}^{0} ( 1 - {E}_{1} ) \qquad [ 14 ]$$

with E₁=e^{-TR/T1} and ε=E₁ cos α. This recurrent equation is a typical first-order nonhomogeneous linear recurrence relation with constant variable. The solution of M_{z}(n) is:

$${M}_{z} ( n ) = ( \begin{matrix}{M}_{p r e p} - {M}^{0} \frac{1 - {E}_{1}}{1 - ε} ) {ε}^{n - 1} + {M}^{0} \frac{1 - {E}_{1}}{1 - ε} , \\ = {M}_{p r e p} \cdot {ε}^{n - 1} + {M}^{0} \frac{1 - {E}_{1}}{1 - ε} ( 1 - {ε}^{n - 1} )\end{matrix} ) \qquad [ 15 ]$$

Therefore, using

$${E}_{2}^{*} = {e}^{- T E / {T}_{2}^{*}} ,$$

the transverse magnetization acquired at the n^{th} gradient echo is given by:

$${M}_{x y} ( n ) = {M}_{z} ( n - 1 ) {E}_{2}^{*} sin α = {M}_{p r e p} \cdot {ε}^{n - I} {E}_{2}^{*} sin α + {M}^{0} \frac{1 - {E}_{1}}{1 - ε} {E}_{2}^{*} sin α ( 1 - {ε}^{n - 1} ) \qquad [ 16 ]$$

It is obvious from Eq. [16] that, when n→∞, the well-known steady-state transverse magnetization (Mₛₛ) for FLASH is:

$${M}_{s s} = {M}_{x y} ( ∞ ) = {M}^{0} \frac{1 - {E}_{1}}{1 - ε} {E}_{2}^{*} sin α \qquad [ 17 ]$$

Eq. [16] can then be expressed as:

$${M}_{x y} ( n ) = {M}_{p r e p} \cdot {ε}^{n - 1} {E}_{2}^{*} sin α + {M}_{s s} \cdot ( 1 - {ε}^{n - 1} ) \qquad [ 18 ]$$

Note that Eq. [18] is consistent with similar derivations in previous work with T₁, T₂ or diffusion preparations.

Hence, the MTF of FLASH with the linear (sequential) and low-high (centric, center k-lines first) profile orders are derived from Eq. [9] and [10], respectively:

$$M T {F}_{F L A S H}^{L N} ( k ) = {M}_{x y} ( ( K + 0.5 ) N + 1 ) = {M}_{p r e p} \cdot {ε}^{( k + 0.5 ) N} {E}_{2}^{*} sin α + {M}_{ss} ( 1 - {ε}^{( k + 0.5 ) N} ) \qquad [ 19 ]$$

$${MTF}_{F L A S H}^{L N} ( k ) = {M}_{x y} ( 2 \vert k \vert N + 1 ) = {M}_{p r e p} \cdot {ε}^{2 \vert k \vert N} {E}_{2}^{*} sin α + {M}_{ss} ( 1 - {ε}^{2 \vert k \vert N} ) \qquad [ 20 ]$$

Corresponding PSFs of the FLASH with the linear and low-high profile orders are derived from Eqs. [11], and [19], [20], respectively:

$$P S {F}_{F L A S H}^{L N} ( z ) = ( {M}_{p r e p} \cdot {E}_{2}^{*} sin α - {M}_{ss} ) \cdot \frac{{ε}^{N} {e}^{i n z} - {e}^{- i n z}}{N ln ε + i 2 π z} + {M}_{ss} \cdot sin c ( πz ) , \qquad [ 21 ]$$

$${PSF}_{F L A S H}^{L N} ( z ) = {\int }_{- 0.5}^{0.5} M T {F}_{F L A S H}^{L N} ( k ) {e}^{i 2 π k z} d k = ( {M}_{p r e p} \cdot {E}_{2}^{*} sin α - {M}_{ss} ) \cdot \\ \frac{{ε}^{N} N ln ε cos ( n z ) + {ε}^{N} π x sin ( π z ) - N ln ε}{{N}^{2} l {n}^{2} ε + {π}^{2} {z}^{1}} + {M}_{s s} \cdot sin c ( πz ) , \qquad [ 22 ]$$

The amplitude-loss effect of a FLASH sequence, characterized with PSF(0), is equivalent for linear
and low-high profile orders. Based on Eq. [21]:

$$P S {F}_{F L A S H}^{L N} ( 0 ) = ( {M}_{p r e p} \cdot {E}_{2}^{*} sin α - {M}_{s s} ) \frac{{ε}^{N} {e}^{0} - {e}^{0}}{N ln ε + 0} + {M}_{s s} \cdot sin c ( 0 ) = \\ ( {M}_{p r e p} \cdot {E}_{2}^{*} sin α - {M}_{s s} ) \frac{{ε}^{N} - 1}{N ln ε + 0} + {M}_{ss} = {M}_{p r e p} \cdot \frac{{ε}^{N} - 1}{N ln ε} {E}_{2}^{*} sin α + {M}_{s s} \cdot ( 1 - \frac{{ε}^{N} - 1}{N ln ε} ) \qquad [ 23 ]$$

Hence PSF(0) and the initial longitudinal magnetization, Mₚᵣₑₚ, obeys a linear relationship with a slope a and an intercept b:

$$P S {F}_{F L A S H} ( 0 ) = {α}_{F L A S H} \cdot {M}_{p r e p} + {b}_{F L A S H} {α}_{F L A S H} = \frac{{ε}^{N} - 1}{N ln ε} {E}_{2}^{*} sin α , {b}_{F L A S H} = {M}_{s s} \cdot ( 1 - \frac{{ε}^{N} - 1}{N ln ε} ) . \qquad [ 24 ]$$

### Numerical Validation

Numerical simulations based on Bloch equations using Matlab (MathWorks, Inc., Natick, MA, USA) were conducted for validation purpose. MTFs of FLASH was generated based on the single-shot FLASH readout with the low-high profile order as used in this study (TR/TE=3.4/2.3 ms, FA=5⁰, N=450;) on its dependence on six Mₚᵣₑₚ values, [0, 0.2, 0.4, 0.6, 0.8, 1.0]. M⁰ is assumed to be 1.0. T₁=1400 ms; T₂*=50 ms.

Infinitely short RF pulses were assumed with instant flipping and no phase or frequency encoding was applied in this simulation. To be aligned with the symmetric MTF in the analytical format, the negative k-space locations were shifted for half a k-space pixel (0.5/N) to compensate for the asymmetric modulation due to interleaved sampling between positive and negative k-space locations. The corresponding PSF are obtained following the Fourier transform of the MTF.

FLASH refers to the acquisition consisting of N excitation pulses with a constant small FA of α, short TR and N PE steps which are centered at the gradient echoes with an echo time of TE. It is assumed that the transverse magnetization before the next FLASH excitation pulses are fully spoiled.

Since the k-space filtering effect due to magnetization relaxation manifests mainly along the slowest PE direction in each echo train (assumed as z-direction), the following derivations are limited in 1D along this direction. The MTF applies a multiplication filter onto the original k-space: S(k)=Sₒᵣᵢ(k)·MTF(k), (1) where k is the k-space location, S(k) is the signal acquired within the k-space, Sₒᵣᵢ(k) is the original signal without magnetization-relaxation induced signal evolution through these k-space locations, which is described as MTF(k).

bSSFP here refers to the acquisition consisting of N alternating excitation pulses with FAs of ±α, short TR and N PE steps which are centered in the middle of the TR with an echo time of TE=TR/2. To smooth the oscillation of the transient magnetization, the bSSFP acquisition is often preceded by a short preparation with an α/2 excitation pulse and TR/2 period. It is assumed that there is ideal dephasing between consecutive excitation pulses and some very small oscillations not fully smoothed by the α/2-TR/2 preparation are neglected. The amplitude effect of a bSSFP sequence, characterized with PSF(0), is equivalent for linear and low-high profile orders.

It should be noted that the pulse sequences, imaging protocols, described herein can be executed with a program(s) fixed on one or more non-transitory computer readable medium. The non-transitory computer readable medium can be loaded onto a computing device, server, imaging device processor, smartphone, tablet, phablet, or any other suitable device known to or conceivable by one of skill in the art.

It should also be noted that herein the steps of the method described can be carried out using a computer, non-transitory computer readable medium, or alternately a computing device, microprocessor, or other computer type device independent of or incorporated with an imaging or signal collection device. An independent computing device can be networked together with the imaging device either with wires or wirelessly. The computing device for executing the present invention can be a completely unique computer designed especially for the implementation of this method. Indeed, any suitable method of analysis known to or conceivable by one of skill in the art could be used. It should also be noted that while specific equations are detailed herein, variations on these equations can also be derived, and this application includes any such equation known to or conceivable by one of skill in the art.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape.

It should be noted that the software associated with the present invention is programmed onto a non-transitory computer readable medium that can be read and executed by any of the computing devices mentioned in this application. The non-transitory computer readable medium can take any suitable form known to one of skill in the art. The non-transitory computer readable medium is understood to be any article of manufacture readable by a computer. Such non-transitory computer readable media includes, but is not limited to, magnetic media, such as floppy disk, flexible disk, hard disk, reel-to-reel tape, cartridge tape, cassette tapes or cards, optical media such as CD-ROM, DVD, Blu-ray, writable compact discs, magneto-optical media in disc, tape, or card form, and paper media such as punch cards or paper tape. Alternately, the program for executing the method and algorithms of the present invention can reside on a remote server or other networked device. Any databases associated with the present invention can be housed on a central computing device, server(s), in cloud storage, or any other suitable means known to or conceivable by one of skill in the art. All of the information associated with the application is transmitted either wired or wirelessly over a network, via the internet, cellular telephone network, RFID, or any other suitable data transmission means known to or conceivable by one of skill in the art.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A method for magnetic resonance imaging of a subject comprising:
a scan, wherein the scan comprises an acquisition scheme, wherein the acquisition scheme of the scan is applied immediately after a saturation module in order to obtain an imaging bias induced by a T₁ relaxation during a long readout.

2. The method of claim 1 wherein the acquisition scheme comprises a 3D FLASH acquisition.

3. The method of claim 1 wherein the acquisition scheme comprises a bSSFP acquisition.

4. The method of claim 1 wherein the imaging bias is from k-space filtering effect induced by the T₁ relaxation.

5. The method of claim 4 wherein the T₁ relaxation is during a long echo train.

6. The method of claim 1 wherein the imaging bias is subtracted using data from other images with pre-selected preparation modules.

7. A method for magnetic resonance imaging of a subject comprising:
a first scan comprising a B₁-insensitive saturation module followed by a fixed saturation delay to generate a consistent magnetization state, a 3D FLASH acquisition with low-high profile ordering, and an RF-prep module with a short hard pulse (FA=α) followed by FLASH readout to generate a FA-encoded image;
a second scan comprising a B₁-insensitive saturation module followed by a fixed saturation delay to generate a consistent magnetization state, a 3D FLASH acquisition with low-high profile ordering, and acquiring a normalizing image without applying the RF-prep module; and
a third scan comprising a 3D FLASH acquisition with low-high profile ordering, wherein the 3D FLASH acquisition is applied immediately after the saturation module in order to obtain an imaging bias induced by a T₁ relaxation during a long readout.

8. The method of claim 7 further comprising subtracting imaging bias from the first and the second scans.

9. The method of claim 7 further comprising conducting an RF-prepared method for FA-encoding and normalizing the first and second scans.

10. The method of claim 7 further comprising the B₁-insensitive saturation module being used to establish Mₙₒᵣₘ after saturation recovery during a fixed delay.

11. The method of claim 7 further comprising using the third scan to obtain intercept, b, without the saturation delay of a normalizing scan.

12. A system for magnetic resonance imaging of a subject comprising:
a magnetic resonance imaging device;
a computer processing device configured for communication and control of the magnetic resonance imaging device, wherein the computer processing device is programmed for executing:
a first scan comprising a B₁-insensitive saturation module followed by a fixed saturation delay to generate a consistent magnetization state, a 3D FLASH acquisition with low-high profile ordering, and an RF-prep module with a short hard pulse (FA=α) followed by FLASH readout to generate a FA-encoded image;
a second scan comprising a B₁-insensitive saturation module followed by a fixed saturation delay to generate a consistent magnetization state, a 3D FLASH acquisition with low-high profile ordering, and acquiring a normalizing image without applying the RF-prep module; and
a third scan comprising a 3D FLASH acquisition with low-high profile ordering, wherein the FLASH acquisition is applied immediately after the saturation module in order to obtain an imaging bias induced by a T₁ relaxation during a long readout.

13. The system of claim 12 further comprising subtracting imaging bias from the first and the second scans.

14. The system of claim 12 further comprising conducting an RF-prepared method for FA-encoding and normalizing the first and second scans.

15. The system of claim 12 further comprising the B₁-insensitive saturation module being used to establish Mₙₒᵣₘ after saturation recovery during a fixed delay.

16. The system of claim 12 further comprising using the third scan to obtain intercept, b, without the saturation delay of a normalizing scan.

17. The system of claim 12 wherein the long readout comprises a long echo train.

18. The system of claim 12 wherein the imaging bias is subtracted using data from other images with pre-selected preparation modules.

19. The system of claim 12 wherein the imaging bias is from a k-space filtering effect induced by the T₁ relaxation.

20. The system of claim 12 further comprising a bSSFP acquisition.
